(19) United States
(12) Patent Application Publication
(10) Pub. No.: US 2018/0015470 A1
(43) Pub. Date: Jan. 18, 2018

(54) MICROFLUIDICS BASED FETAL CELL DETECTION AND ISOLATION FOR NON-INVASIVE PRENATAL TESTING

(71) Applicants: Fanqing Chen, Moraga, CA (US); Unimed Biotech (Shanghai) Co., Ltd., Shanghai (CN)

(72) Inventors: Fanqing Chen, Moraga, CA (US); Tania Chakrabarty, Dublin, CA (US)

(21) Appl. No.: 15/545,604

(22) PCT Filed: Jan. 19, 2016
(86) PCT No.: PCT/US2016/013865

§ 371 Date: Jul. 21, 2017

Related U.S. Application Data
(60) Provisional application No. 62/107,261, filed on Jan. 23, 2015.

(51) Int. Cl. B01L 3/00; C12Q 1/68

(52) U.S. Cl. CPC B01L 3/502753; C12Q 1/6883; B01L 2200/0668; B01L 2300/0681; C12Q 2600/158

(57) ABSTRACT

Embodiments disclosed herein provides methods for isolation of fetal cells for non-invasive prenatal diagnosis, comprising: providing a maternal blood sample; applying the maternal blood sample to a filter integrated on a microfluidic device to thereby enrich the nucleated blood cells from the maternal blood sample; labeling the enriched nucleated blood cells, within the microfluidic device, with a fluorescent binding moiety or affinity molecule that specifically binds to a fetal cell-specific antigen or a non-fetal cell-specific antigen; and isolating the fetal cells. Embodiments disclosed herein provide integrated microfluidic devices for non-invasive isolation of fetal cells, comprising: a filter; a binding moiety or affinity molecule that specifically binds to a fetal cell-specific antigen or a non-fetal cell-specific antigen; and a microscopy-visualizable chamber.

### CROSS-REFERENCE TO RELATED APPLICATIONS

This application claims priority to U.S. Provisional Application No. 62/107,261, filed Jan. 23, 2015, entitled “MICROFLUIDICS BASED FETAL CELL DETECTION AND ISOLATION FOR NON-INVASIVE PRENATAL TESTING,” the content of which is hereby incorporated by reference in its entirety.

### BACKGROUND

### Field of the Invention

The embodiments disclosed herein relate to methods, devices and kits for microfluidics based fetal cell detection and isolation from maternal blood for non-invasive prenatal diagnostics.

### Description of the Related Art

Fetal cell isolation from maternal blood for non-invasive prenatal diagnosis presents various challenges due to the rarity of such cells. Various approaches have been attempted to extract and analyze such cells for downstream genetic analysis and diagnostic assays but the success and purity of such extraction has been very poor. Additionally, the throughput of such detection and extraction outside of FACS-based analysis has remained low, presenting another challenge in the field of non-invasive prenatal testing. Hence non-invasive prenatal diagnostics till date has mostly relied on analysing cell-free DNA (cfDNA) from maternal blood. But cfDNA being highly fragmented presents many challenges in analyzing that DNA and presenting a complete prenatal analysis of fetal genome anomalies when present.

### SUMMARY

Embodiments disclosed herein provides methods for isolation of fetal cells for non-invasive prenatal diagnosis, comprising: providing a maternal blood sample; applying the maternal blood sample to a filter integrated on a microfluidic device to thereby enrich the nucleated blood cells from the maternal blood sample; labeling the enriched nucleated blood cells, within the microfluidic device, with a fluorescent binding moiety or affinity molecule that specifically binds to a fetal cell-specific antigen or a non-fetal cell-specific antigen; and isolating the fetal cells. In some embodiments, the filter is transparent. In some embodiments, the nucleated blood cells are enriched via morphology and/or other physical characteristics of the cells. In some embodiments, the methods further comprise visualizing the labeled nucleated blood cells in a microscopy-visualizable chamber within the microfluidic device. In some embodiments, the methods further comprise selectively immobilizing labeled nucleated blood cells within the filter fitted microfluidic device for visualization and/or microscopic analysis. In some embodiments, the visualization and/or microscopic analysis is manual. In some embodiments, the visualization and/or microscopic analysis is automated via machine vision. In some embodiments, the fetal cells are nucleated red blood cells (nRBCs). In some embodiments, the fetal cell-specific antigen is selected form the group consisting of CD45, transferin receptor (CD71), glycophorin A (GPA), HLA-G, EGFR, thrombospondin receptor (CD36), CD34, HbF, HAE 9, FB3-2, H3-3, erythropoietin receptor, HBE, AFP, APOC3, SERPINC1, AMBP, CPB2, ITIH1, APOH, HPX, beta-hCG, AHSG, APOB, J42-4-d, 2,3-biophosphoglycerate (BPG), Carbonic anhydrase (CA), Thymidine kinase (TK), MMP14 (matrix metalloproteinase 14), and fetal hemoglobin. In some embodiments, the filter is configured to enrich nucleated blood cells and/or remove mature red blood cells (RBCs). In some embodiments, the methods further comprise removing non-fetal cells. In some embodiments, the removing non-fetal cells comprises immobilizing the non-fetal cells. In some embodiments, the methods further comprise immobilizing the fetal cells. In some embodiments, the immobilizing the fetal cells comprises contacting the fetal cells with a binding moiety or affinity molecule that specifically binds to a fetal cell-specific antigen. In some embodiments, the fetal cell-specific antigen is selected form the group consisting of CD45, transferin receptor (CD71), glycophorin A (GPA), EGFR, thrombospondin receptor (CD36), CD34, HbF, HAE 9, FB3-2, H3-3, erythropoietin receptor, HBE, AFP, APOC3, SERPINC1, AMBP, CPB2, ITIH1, APOH, HPX, beta-hCG, AHSG, APOB, J42-4-d, 2,3-biophosphoglycerate (BPG), Carbonic anhydrase (CA), Thymidine kinase (TK), MMP14 (matrix metalloproteinase 14), and fetal hemoglobin. In some embodiments, the methods further comprise analyzing the isolated fetal cell for nucleic acid sequence via sequencing or detecting genetic abnormalities in the isolated fetal cell using other commonly used methods such as FISH or DNA microarray. In some embodiments, the analyzing a nucleotide sequence of a nucleic acid molecule comprises hybridizing a detectable probe to the genomic DNA of one or more isolated fetal cells. In some embodiments, the analyzing a nucleotide sequence of a nucleic acid molecule comprises sequencing genomic DNA of one or more isolated fetal cells. In some embodiments, the sequencing genomic DNA comprises sequencing the DNA of a single cell, and wherein sequencing the DNA of a single cell is performed for one or more isolated fetal cells. In some embodiments, the analyzing expression of a gene comprises hybridizing a detectable antibody to the surface of one or more isolated fetal cells. In some embodiments, the isolated fetal cells are analyzed for a genetic defect.

Embodiments disclosed herein provide integrated microfluidic devices for non-invasive isolation of fetal cells, comprising: a filter; a binding moiety or affinity molecule that specifically binds to a fetal cell-specific antigen or a non-fetal cell-specific antigen; and a microscopy-visualizable chamber. In some embodiments, the integrated microfluidic devices further comprise a reagent that is configured to detect one or more nucleotide sequences of the isolated fetal cells.

Embodiments disclosed herein further provide kits comprising: an integrated microfluidic device for non-invasive isolation of fetal cells, comprising: a filter; a binding moiety or affinity molecule that specifically binds to a fetal cell-specific antigen or a non-fetal cell-specific antigen; and a microscopy-visualizable chamber, and a reagent that is configured to detect one or more nucleotide sequences of the isolated fetal cells.

### BRIEF DESCRIPTION OF THE DRAWINGS

FIG. 1 depicts an exemplary process for isolating fetal cells from a maternal blood sample in one embodiment.

FIG. 2 depicts an exemplary process for downstream analysis of isolated fetal cells.

### DETAILED DESCRIPTION OF THE PREFERRED EMBODIMENT

Methods and devices disclosed herein for fetal cell isolation combine affinity and/or biomarker based isolation with morphology-based isolation. By combining these processes on an integrated microfluidic device, the methods and devices disclosed herein resolve the long-standing challenge of throughput for fetal cell isolation from maternal blood samples. Unlike FACS, disclosed herein are visualization-based methods similar to imaging cytometry that are performed on a microscope platform. The methods described here can be partially or fully automated which adds another benefit to the current disclosure.

Preferably, the methods and devices disclosed herein take advantage of a filter integrated on a microfluidic chip. This is used at an early step of the isolation process which allows for enrichment of nucleated blood cells from maternal blood samples. For example, the morphology-based selection filter allows most or all of the mature red blood cells (RBCs) to pass through the openings on the filter and captures most of the nucleated blood cells. The nucleated blood cells are then stained and/or labeled with nuclear stain and/or specific biomarkers for positive or negative selection of a subset of nucleated blood cells of interest. Of particular interest for non-invasive prenatal diagnostics is the population of fetal nucleated red blood cells (fnRBC).

As such, the methods and devices disclosed herein permit: 1) cell filtration, staining, enrichment (if needed) on one integrated platform minimizing manual labor and intervention; 2) use of automation to streamline processing of maternal blood; 3) use of microfluidics for reagent delivery providing reduction in reagent use and cost; 4) use of sealed microfluidic chamber reducing contamination and sample mix-up; and 5) a flexible platform that can be used for other functionalities such as cell lysis.

### Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a”, “an”, and “the” include plural references unless indicated otherwise. For example, “a” dimer includes one or more dimers.

As used herein, the term “microfluidic device” generally refers to a device through which materials, particularly fluid borne materials, such as liquids, can be transported, in some embodiments on a micro-scale, and in some embodiments on a nanoscale. Thus, the microfluidic devices described by the presently disclosed subject matter can comprise microscale features, nanoscale features, and combinations thereof. The samples delivered on such a device may be fluids alone or fluids with suspended components such as cells and particles.

Accordingly, an exemplary microfluidic device typically comprises structural or functional features dimensioned on the order of a millimeter-scale or less, which are capable of manipulating a fluid at a flow rate on the order of 5 mL/min or less. Typically, such features include, but are not limited to channels, fluid reservoirs, reaction chambers, mixing chambers, and separation regions. In some examples, the channels include at least one cross-sectional dimension that is in a range of from about 0.1 μm to about 10 millimeters. The use of dimensions on this order allows the incorporation of a greater number of channels in a smaller area, and utilizes smaller volumes of fluids.

A microfluidic device can exist alone or can be a part of a microfluidic system which, for example and without limitation, can include: pumps and valves for introducing fluids, e.g., samples, reagents, buffers and the like, into the system and/or through the system; detection equipment or systems; data storage systems; and control systems for controlling fluid transport and/or direction within the device, monitoring and controlling environmental conditions to which fluids in the device are subjected, e.g., temperature, current, and the like using sensors where applicable. The valves in such system may be pressure or vacuum driven.

As used herein, the terms “channel,” “micro-channel,” and “microfluidic channel” are used interchangeably and can mean a recess or cavity formed in a material by imparting a pattern from a patterned substrate into a material or by any suitable material removing technique, or can mean a recess or cavity in combination with any suitable fluid-conducting structure mounted in the recess or cavity, such as a tube, capillary, or the like.

As used herein, the terms “flow channel” and “control channel” are used interchangeably and can mean a channel in a microfluidic device in which a material, such as a fluid, e.g., a gas or a liquid, can flow through. More particularly, the term “flow channel” refers to a channel in which a material of interest, e.g., any fluid (with or without suspended materials) or a chemical reagent, can flow through. Further, the term “control channel” refers to a flow channel in which a material, such as a fluid, e.g., a gas or a liquid, can flow through in such a way to actuate a valve or pump. The fluid flow in such channels can be pressure or vacuum driven for active flow or passively driven via surface tension.

As used herein, “chip” refers to a solid substrate with a plurality of one-, two- or three-dimensional micro structures or micro-scale structures on which certain processes, such as physical, chemical, biological, biophysical or biochemical processes, etc., can be carried out. The micro structures or micro-scale structures such as, channels and wells, electrode elements, electromagnetic elements, are incorporated into, fabricated on or otherwise attached to the substrate for facilitating physical, biophysical, biological, biochemical, chemical reactions or processes on the chip. The chip may be thin in one dimension and may have various shapes in other dimensions, for example, a rectangle, a circle, an ellipse, or other irregular shapes. The size of the major surface of chips of the present invention can vary considerably, e.g., from about 1 mm² to about 0.25 m². Preferably, the size of the chips is from about 4 mm² to about 25 cm² with a characteristic dimension from about 1 mm to about 5 cm. The chip surfaces may be flat, or not flat. The chips with non-flat surfaces may include channels or wells fabricated on the surfaces.

A microfluidic chip can be made from any suitable materials, such as PDMS (Polydimethylsiloxane), glass, PMMA (polymethylmethacrylate), PET (polyethylene terephthalate), PC (Polycarbonate), etc., or a combination thereof. The filter integrated in the chip may be made from similar materials or different materials.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule, and can be an immunoglobulin of any class, e.g., IgG, IgM, IgA, IgD and IgE. IgY, which is the major antibody type in avian species such as chicken, is also included within the definition. As used herein, the term encompasses not only intact polyclonal or monoclonal antibodies, but also fragments thereof (such as Fab, Fab′, F(ab′)2, Fv), single chain (ScFv), mutants thereof, naturally occurring variants, fusion proteins comprising an antibody portion with an antigen recognition site of the required specificity, humanized antibodies, chimeric antibodies, and any other modified configuration of the immunoglobulin molecule that comprises an antigen recognition site of the required specificity.

As used herein, the term “specifically binds” refers to the binding specificity of a specific binding pair. Recognition by an antibody of a particular target in the presence of other potential targets is one characteristic of such binding. Specific binding involves two different molecules wherein one of the molecules specifically binds with the second molecule via chemical or physical means. The two molecules are related in the sense that their binding with each other is such that they are capable of distinguishing their binding partner from other assay constituents having similar characteristics. The members of the binding component pair are referred to as ligand and receptor (anti-ligand), specific binding pair (SBP) member and SBP partner, antibody-antigen and the like. A molecule may also be an SBP member for an aggregation of molecules; for example an antibody raised against an immune complex of a second antibody and its corresponding antigen may be considered to be an SBP member for the immune complex.

It is understood that aspects and embodiments of the invention described herein include “consisting” and/or “consisting essentially of” aspects and embodiments.

Other objects, advantages and features of the present invention will become apparent from the following specification taken in conjunction with the accompanying drawings.

### Method for Isolating Fetal Cells

Embodiments disclosed herein provide methods for isolation of fetal cells for non-invasive prenatal diagnosis. The fetal cells will be isolated from a biological sample, for example, a maternal blood sample. In some embodiments, the methods may comprise applying a maternal blood sample to a filter integrated on a microfluidic device to thereby enrich the nucleated blood cells from the maternal blood sample. In some embodiments, the methods may comprise labeling the enriched nucleated blood cells, for example, within the microfluidic device, with a fluorescent binding moiety or affinity molecule that specifically binds to a fetal cell-specific antigen or a non-fetal cell-specific antigen for positive selection or negative selection.

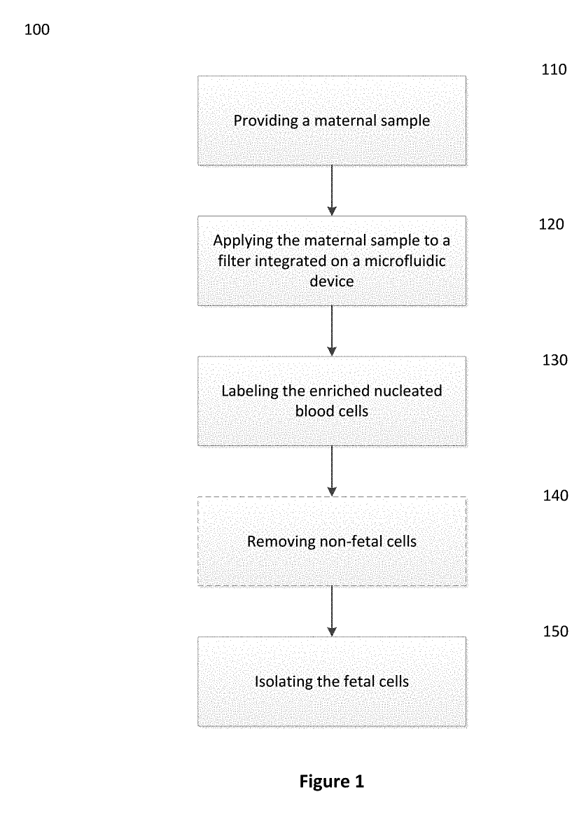

A non-limiting example of the method 100 for isolating fetal cells in accordance with the disclosed embodiments is illustrated in the flow diagram shown in FIG. 1. As illustrated in FIG. 1, the method 100 can include one or more functions, operations or actions as illustrated by one or more operations 110-150.

Method 100 can begin at operation 110, “Providing a maternal sample.” Operation 110 can be followed by operation 120, “Applying the maternal sample to a filter integrated on a microfluidic device.” Operation 120 can be followed by operation 130, “Labeling the enriched nucleated blood cells.” Operation 130 can be followed by optional operation 140, “Removing non-fetal cells.” Operation 130 or operation 140 can be followed by operation 150, “Isolating the fetal cells.”

In FIG. 1, operations 110-150 are illustrated as being performed sequentially with operation 110 first and operation 150 last. It will be appreciated, however, that these operations can be combined and/or divided into additional or different operations as appropriate to suit particular embodiments. For example, additional operations can be added before, during or after one or more operations 110-150. In some embodiments, one or more of the operations can be performed at about the same time. In some embodiments, the method only consists of operations 110, 120, 130 and 150 but not any other operations. In some embodiments, the method consists essentially of operations 110, 120, 130 and 150. In some embodiments, the method only consists of operations 110, 120, 130 and 150 and operation 140, but not any other operations.

At operation 110, “Providing a maternal sample,” maternal samples containing one or more nucleated fetal cells can be obtained from human pregnant mothers using standard blood draw. The maternal sample can be taken during the first trimester (about the first three months of pregnancy), the 2nd trimester (about months 4-6 of pregnancy), or the third trimester (about months 7-9 of pregnancy). Typically, the sample obtained is a blood sample.

When obtaining a maternal sample from human (e.g., blood sample), the amount of sample can vary depending upon size, gestation period, and the condition being screened, in one embodiment, up to 200, 175, 150, 125, 100, 90, 80, 70, 60, 50, 40, 30, 20, 10, or 5 mL of a sample is obtained. In one embodiment, 5-200, 10-100, or 30-50 mL of sample is obtained. In one embodiment, more than 1, 2, 3, 4, 5, 10, 20, 30, 40, 50, 60, 70, 80, 90, 100 or 150 mL of a sample is obtained. In one embodiment between about 10-100 or 30-50 ml of a peripheral blood sample is obtained from a pregnant female. In some embodiments, a blood sample is obtained from a pregnant human mother within 36, 24, 22, 20, 18, 16, 14, 12, 10, 8 weeks of conception, or a range between any of the above values. For example, a blood sample is obtained from a pregnant human mother as early as 8 weeks of conception. In some embodiments, a blood sample is obtained from a pregnant human mother even after a pregnancy has terminated.

The sample is subjected to one or more steps that enrich the nucleated fetal cells relative to the total components of the sample and/or enrich the nucleated fetal cells relative to the total cells in the sample. The maternal sample can be used as is, pre-diluted in desirable buffer or diluted on chip if required prior to applying it to a filter.

In some embodiments, enrichment of nucleated fetal cells occurs using one or more size-based separation methods. Examples of size-based separation modules include filtration membranes, molecular sieves, and matrixes. Examples of size-based separation modules contemplated by the present invention include those disclosed in International Publication No. WO 2004/113877, which is herein incorporated by reference in its entirety. Other size based separation methods are disclosed in International Publication No. WO 2004/0144651 and U.S. Patent Application Publication Nos. US20080138809A1 and US20080220422A1, which are herein incorporated by reference in their entireties.

At operation 120, “Applying the maternal sample to a filter integrated on a microfluidic device,” any filter that is suitable to select nucleated blood cells and/or mature red blood cells (RBC) may be used. In some embodiments, a filter may comprise openings that have a size and/or shape which allows the RBC to pass through, but retains the nucleated blood cells. For example, the openings may be a size that is, is about, or is less than, 4.0 μm, 4.1 μm, 4.2 μm, 4.3 μm, 4.4 μm, 4.5 μm, 4.6 μm, 4.7 μm, 4.8 μm, 4.9 μm, 5.0 μm, 6.0 μm, 7.0 μm, 8.0 μm, 9.0 μm, 10.0 μm, 11.0 μm, 12.0 μm, 13.0 μm, 14.0 μm, 15.0 μm, 16.0 μm, 17.0 μm, 18.0 μm, 19.0 μm, 20.0 μm, 21.0 μm, 22.0 μm, 23.0 μm, 24.0 μm, 25.0 μm, 26.0 μm, 27.0 μm, 28.0 μm, 29.0 μm, 30.0 μm, or a range between any two of the above values, for example, between 2.0 μm to 2.5 μm, between 1.8 μm to 3.0 μm, etc. In some embodiments, the shape of the openings may be rectangular, circular, oval, triangular, etc., or an irregular shape. As used herein, the “size” of the openings refers the smallest effective opening for the filter. Accordingly, in some embodiments, nucleated blood cells may be enriched when RBCs pass through openings on a filter having a size and/or shape that allows RBCs to pass through, but not nucleated blood cells.

In some embodiments, a filter may be coated with a binding moiety or affinity molecule that selectively binds nucleated blood cells or RBCs. For example, an antibody that specifically binds to nucleated blood cells may be used to coat the filter, so that nucleated blood cells are retained while the RBCs pass through the filter.

In some embodiments, even an enriched product can be dominated (>50%) by cells not of interest (e.g., nucleated maternal red blood cells). In some cases, the nucleated fetal cells of an enriched sample makes up at least 2, 3, 4, 5, 10, 20, 30, 40, 50, 60, 70, 80, 90 or 95% of all cells in the enriched sample. For example, using the methods and systems described herein, a maternal blood sample of 10-20 mL from a pregnant human can be enriched for one or more nucleated fetal cells, such as nucleated red blood cells, such that the enriched sample has a total of about 1 thousand to about 10 million cells, 2% of which are nucleated fetal cells and the rest of the cells are maternal. In sonic embodiments, the enrichment steps performed have removed at least 50, 60, 70, 80, 85, 90, 91, 92, 93, 94, 95, 96, 97, 98, 99, 99.5, 99.6, 99.7, 99.8 or 99.9% of all unwanted analytes (e.g., maternal cells such as platelets and leukocytes, mature RBCs) from a sample.

At operation 130, “Labeling the enriched nucleated blood cells,” the enriched nucleated blood cells may be labeled, directly or indirectly, with a dye. In some embodiments, a dye that stains DNA, such as Acridine orange (AO), ethidium bromide, hematoxylin, Nile blue, Hoechst, Safranin, or DAPI, may be used. In some embodiments, a cell type-specific dye, for example, a dye that specifically labels a fetal cell or a non-fetal cell, may be used. The cell type-specific dye may be used to label the cells directly or indirectly, for example, through a cell type-specific antibody. The labeling strategy involved may be sequentially carried out or simultaneously carried out.

In some embodiments, an optional operation 140, “Removing non-fetal cells,” may be performed to enrich the fetal cells. At this optional operation, non-fetal cells may be removed by a variety of techniques from the enriched nucleated blood cells. For example, in some embodiments, the bulk of the non-fetal cells may be removed by differential lysis to lyse the mature RBCs or density gradient centrifugation to enrich for the nucleated fraction or by immobilizing the non-fetal cells in a micro-channel or chamber on the microfluidic device that is different from the micro-channel or chamber the fetal cells are located. These enrichment steps can use one or a combination of the above mentioned techniques. Partial enrichment steps can also be carried out off chip before enriched sample is loaded on chip for fetal cell isolation. For example, the non-fetal cells may be immobilized in a micro-channel or chamber coated with a binding moiety or affinity molecule that specifically binds to the non-fetal cells. In some embodiments, the non-fetal cells may be removed by a fluorescence-activated process.

Affinity-Based Enrichment

In some embodiments, nucleated fetal cells can be enriched based on their affinity for a binding moiety or affinity molecule. In such embodiments, the binding moiety is suitably labeled to facilitate separation of the nucleated fetal cells from undesired components of a maternal sample. For example, a binding moiety with affinity for a nucleated fetal cell can bind the nucleated fetal cell and can be used to separate nucleated fetal cells by being bound to a solid support such as a magnetic bead or a non-magnetic solid phase of a chromatographic material, or the binding moiety can be detectably labeled such that the nucleated fetal cells can be distinguished from other sample components by detection-assisted enrichment of the nucleated fetal cells, visualization based or otherwise.

In some embodiments, the affinity methods include using a directly or indirectly labeled binding moiety or affinity molecule having affinity for a fetal cell surface marker. For example, a binding moiety or affinity molecule can be attached to a stationary phase, a fluorophore, a radionuclide, or other detectable moiety, and the sample can be contacted with the labeled binding moiety or affinity molecule under conditions that allow the fetal nucleated cells to be specifically bound to the binding moiety or affinity molecule while other components of the sample do not specifically bind to the binding moiety or affinity molecule. The labeled binding moiety or affinity molecule can then be treated, for example using flow cytometry, imaging cytometry, micromanipulator, optical tweezers, DEP, magnetic capture, density centrifuge, and size-based liquid chromatography, to separate components of the maternal sample bound to the labeled binding moiety or affinity molecule from components of the maternal sample not bound to the labeled binding moiety or affinity molecule. The bound components of the maternal sample can optionally be washed to remove non-specifically bound components. The components of the sample bound to the labeled binding moiety or affinity molecule, which includes fetal nucleated cells, can then be retained or harvested for further enrichment or for analysis.

Binding moieties can include e.g., proteins, nucleic acids, and carbohydrates that specifically bind to nucleated fetal cells. In one embodiment, the binding moiety has affinity for one or more carbohydrates, such as galactose. For example, a binding moiety can be lectin. In other embodiments, the binding moiety is an antibody. Examples of such binding moiety antibodies include: anti-matrix metalloproteinase 14 (anti-MMP14), anti-transferin receptor (anti-CD71), anti-glycophorin A (anti-GPA), anti-thrombospondin receptor (anti-CD36), anti-CD34, anti-HAE9, anti-FB3-2, anti-H3-3, anti-erythropoietin receptor, anti-CD235a, anti-carbohydrates, anti-selectin, anti-CD45, anti-GPA, anti-antigen-i, anti-EpCAM, anti-E-cadherin, anti-Muc-1, anti-hPL, anti-CHS2, anti-KISS1, anti-GDF15, anti-TFP12, anti-CGB, anti-LOC90625, anti-FN1, anti-COL1A2, anti-PSG9, anti-PSG1, anti-HBE, anti-AFP, anti-APOC3, anti-SERPINC1, anti-AMBP, anti-CPB2, anti-APOH, anti-HPX, anti-beta-hCG, anti-AHSG, anti-APOB, anti-J42-4-d, anti-2,3-biophosphoglycerate (anti-BPG), anti-Carbonic anhydrase (anti-CA), or anti-Thymidine kinase (anti-TK).

In one embodiment, a nucleated fetal cell is enriched using anti-MMP14, anti-CD71 and/or anti-GPA selection. In another embodiment, nucleated fetal cells are enriched using one or more antibodies or antibody fragments that can bind a protein expressed from the genes MMP14, CD71, GPA, HLA-G, EGFR, CD36, CD34, HbF, HAE 9, FB3-2, H3-3, erythropoietin receptor, HBE, AFP, APOC3, SERPINC1, AMBP, CPB2, ITIH1, APOH, HPX, beta-hCG, AHSG, APOB, J42-4-d, BPG, CA, or TK.

Stationary Phase-Based Enrichment

In some embodiments, affinity chromatographic methods can be used. For example, a binding moiety or affinity molecule can be attached to a stationary phase, such as a bead, column or particle, and the sample can be contacted with the affinity molecule-attached stationary phase under conditions that allow the fetal nucleated cells to be specifically bound to the binding moiety or affinity molecule while other components of the sample do not specifically bind to the binding moiety or affinity molecule. The contacted stationary phase can then be treated, for example, using a mobile phase, to separate components of the maternal sample bound to the affinity molecule-attached stationary phase from components of the maternal sample not bound to the affinity molecule-attached stationary phase. The components of the sample bound to the affinity molecule-attached stationary phase, which includes fetal nucleated cells, can then be retained or harvested for further enrichment or for analysis.

In one embodiment, a magnetic particle is used to enrich nucleated fetal cells. In one embodiment, a binding moiety such as an antibody can be coupled to a magnetic particle (e.g., a magnetic bead). In one embodiment the bead is couple to an antibody or fragment of an antibody that is an anti-MMP14, anti-CD71, anti-GPA, anti-CD36, anti-CD34, anti-HbF, anti-HAE9, anti-FB3-2, anti-H3-3, anti-erythropoietin receptor, anti-CD235a, anti-carbohydrates, anti-selectin, anti-CD45, anti-GPA, anti-antigen-i, anti-EpCAM, anti-E-cadherin, anti-Muc-1, anti-hPL, anti-CHS2, anti-KISS1, anti-GDF15, anti-CRH, anti-TFP12, anti-CGB, anti-LOC90625, anti-COL1A2, anti-PSG9, anti-PSG1, anti-HBE, anti-AFP, anti-APOC3, anti-SERPINC1, anti-AMBP, anti-CPB2, anti-ITIH1, anti-APOH, anti-HPX, anti-beta-hCG, anti-AHSG, anti-APOB, anti-J42-4-d, anti-BPG, anti-CA, or anti-TK antibody or fragment of an antibody.

Any of a variety of fluorescent molecules or dyes that can be used with the nucleic acid, antibody or antibody-based fragment probes provided herein, including, but not limited to, Alexa Fluor 350, AMCA, Alexa Fluor 488, Fluorescein isothiocyanate (FITC), GFP, RFP, YFP, BFP, CFSE, CFDA-SE, DyLight 288, SpectrumGreen, Alexa Fluor 532, Rhodamine, Rhodamine 6G, Alexa Fluor 546, Cy3 dye, tetramethylrhodamine (TRITC), SpectrumOrange, Alexa Fluor 555, Alexa Fluor 568, Lissamine rhodamine B dye, Alexa Fluor 594, Texas Red dye, SpectrumRed, Alexa Fluor 647, Cy5 dye, Alexa Fluor 660, Cy5.5 dye, Alexa Fluor 680, Phycoerythrin (PE), Propidium iodide (PI), Peridinin chlorophyll protein (PerCP), PE-Alexa Fluor 700, PE-Cy5 (TRI-COLOR), PE-Alexa Fluor 750, PE-Cy7, APC, APC-Cy7, Draq-5, Pacific Orange, Amine Aqua, Pacific Blue, Alexa Fluor 405, Alexa Fluor 430, Alexa Fluor 500, Alexa Fluor 514, Alexa Fluor-555, Alexa fluor-568, Alexa Fluor-610, Alexa Fluor-633, DyLight 405, DyLight 488, DyLight 549, DyLight 594, DyLight 633, DyLight 649, DyLight 680, DyLight 750, or DyLight 800.

Fetal Biomarkers

In some embodiments fetal biomarkers can be used to detect and/or isolate one or more fetal cells. For example, this can be performed by distinguishing between fetal and maternal nucleated cells based on relative expression of a gene (e.g., DYS1, DYZ, CD-71, MMP14) that is differentially expressed during fetal development. In one embodiment of the provided invention, detection of transcript or protein expression of one or more genes including, MMP14, CD71, GPA, HLA-G, EGFR, CD36, CD34, HbF, HAE 9, FB3-2, H3-3, erythropoietin receptor, HBE, AFP, APOC3, SERPINC1, AMBP, CPB2, ITIH1, APOH, HPX, beta-hCG, AHSG, APOB, J42-4-d, 2,3-biophosphoglycerate (BPG), Carbonic anhydrase (CA), or Thymidine kinase (TK), is used to enrich, purify, enumerate, identify detect or distinguish a fetal cell. The expression can include a transcript expressed from these genes or a protein. In one embodiment of the provided invention, expression of one or more genes including MMP14, CD71, GPA, EGFR, CD36, CD34, HbF, RAE 9, FB3-2, H3-3, erythropoietin receptor, HBE, AFP, AHSG, J42-4-d, BPG, CA, or TK, is used to identify, purify, enrich, or enumerate a nucleated fetal cell such as a nucleated fetal red blood cell.

Beta-hCG (also known as h-hCG, HCG, CGB, CGB3 and hCGB) is a member of the glycoprotein hormone beta chain family and encodes the beta 3 subunit of chorionic gonadotropin (CG). Glycoprotein hormones are heterodimers consisting of a common alpha subunit and an unique beta subunit which confers biological specificity. CG is produced by the trophoblastic cells of the placenta and stimulates the ovaries to synthesize the steroids that are essential for the maintenance of pregnancy. The beta subunit of CG is encoded by 6 genes which are arranged in tandem and inverted pairs on chromosome 19q13.3 and contiguous with the luteinizing hormone beta subunit gene.

APOB (also known as apolipoprotein B (including Ag(x) antigen) and FLDB) is the main apolipoprotein of chylomicrons and low density lipoproteins. It occurs in plasma as two main isoforms, apoB-48 and apoB-100: the former is synthesized exclusively in the gut and the latter in the liver. The intestinal and the hepatic forms of apoB are encoded by a single gene from a single, very long mRNA. The two isoforms share a common N-terminal sequence. The shorter apoB-48 protein is produced after RNA editing of the apoB-100 transcript at residue 2180 (CAA→UAA), resulting in the creation of a stop codon, and early translation termination. Mutations in this gene or its regulatory region cause hypobetalipoproteinemia, nortnotriglyceridemic hypobetalipoproteinemia, and hypercholesterolemia due to ligand-defective apoB, diseases affecting plasma cholesterol and apoB levels.

AHSG (also known as alpha-2-HS-glycoprotein; AHS; A2HS; HSGA; and ETUA) is a glycoprotein present in the serum and can be synthesized by hepatocytes. The AHSG molecule consists of two polypeptide chains, which are both cleaved from a proprotein encoded from a single mRNA. It is involved in several functions, such as endocytosis, brain development and the formation of bone tissue. The protein is commonly present in the cortical plate of the immature cerebral cortex and bone marrow hemopoietic matrix, and it has therefore been postulated that it participates in the development of the tissues.

HPX (also known as hemopexin) can bind heme. It can protect the body from the oxidative damage that can be caused by free heme by scavenging the heme released or lost by the turnover of heme proteins such as hemoglobin. To preserve the body's iron, upon interacting with a specific receptor situated on the surface of liver cells, hemopexin can release its bound ligand for internalisation.

CPB2 (also known as carboxypeptidase B2 (plasma); CPU; PCPB; and TAFI) is an enzyme that can hydrolyze C-terminal peptide bonds. The carboxypeptidase family includes metallo-, serine, and cysteine carboxypeptidases. According to their substrate specificity, these enzymes are referred to as carboxypeptidase A (cleaving aliphatic residues) or carboxypeptidase B (cleaving basic amino residues). The protein encoded by this gene is activated by trypsin and acts on carboxypeptidase B substrates. After thrombin activation, the mature protein downregulates fibrinolysis. Polymorphisms have been described for this gene and its promoter region. Available sequence data analyses indicate splice variants that encode different isoforms.

ITIH1 (also known as inter-alpha (globulin) inhibitor H1; H1P; ITIH; LATIH; and MGC126415) is a serine protease inhibitor family member. It is assembled from two precursor proteins: a light chain and either one or two heavy chains. ITIH1 can increase cell attachment in vitro.

APOH (also known as apolipoprotein H (beta-2-glycoprotein I); BG; and B2G1) has been implicated in a variety of physiologic pathways including lipoprotein metabolism, coagulation, and the production of antiphospholipid autoantibodies. APOH may be a required cofactor for anionic phospholipid binding by the antiphospholipid autoantibodies found in sera of many patients with lupus and primary antiphospholipid syndrome.

AMBP (also known as alpha-1-microglobulin/bikunin precursor; HCP; ITI; UTI; EDC1; HI30; ITIL; IATIL; and ITILC) encodes a complex glycoprotein secreted in plasma. The precursor is proteolytically processed into distinct functioning proteins: alpha-1-microglobulin, which belongs to the superfamily of lipocalin transport proteins and may play a role in the regulation of inflammatory processes, and bikunin, which is a urinary trypsin inhibitor belonging to the superfamily of Kunitz-type protease inhibitors and plays an important role in many physiological and pathological processes. This gene is located on chromosome 9 in a cluster of lipocalin genes.

J42-4-d is also known as t-complex 11 (mouse)-like 2; MGC40368 and TCP11L2.

At operation 150, “Isolating the fetal cells,” the fetal cells may be isolated using either manual or automated methods. For example, the fetal cells may be isolated by selection of fetal cells and/or deselection of non-fetal cells. In some embodiments, the fetal cells are isolated by single cell capture. In some embodiments, the fetal cells may be isolated while being visualized on a microscope platform.

In various embodiments, at certain point before, during or after an operation, a wash buffer may be flowed to the micro-channels and/or chambers to wash off excess reagents, such as buffers, dyes, antibodies, nucleic acids, cells, cell debris, etc.

Assessing the Isolated Fetal Cells

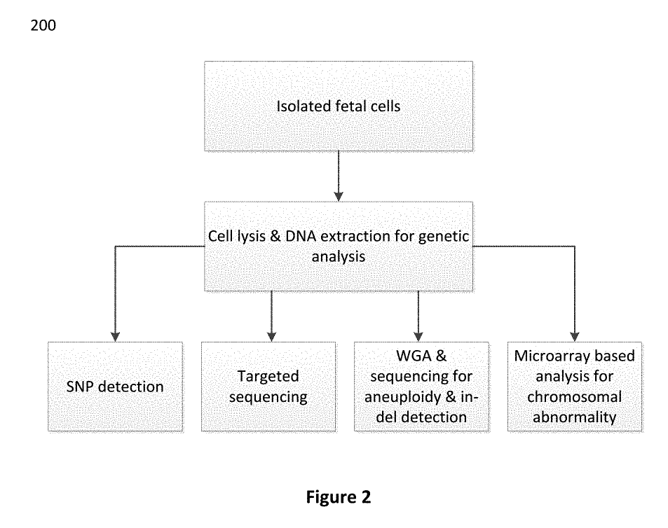

The embodiments disclosed herein further provide the flexibility for performing one or more analyses on the isolated fetal cells for prenatal testing and/or diagnostics. A non-limiting example of the downstream analysis 200 of isolated fetal cells in accordance with the disclosed embodiments is illustrated in the diagram shown in FIG. 2. In FIG. 2, the isolated fetal cells may be subject to steps of cell lysis and DNA extraction for the downstream genetic analysis. In some embodiments, the cell lysis and/or DNA extraction may be conducted on the same or a separate microfluidic chip which fits the cell sorting chip. In some embodiments, the cell lysis and/or DNA extraction may be conducted using standard approaches which may not be chip based.

In some embodiments, the isolated fetal cells can be assessed for a nucleotide sequence of a nucleic acid molecule or expression of a gene. For example, the isolated fetal cells may be analyzed for a genetic defect, such as SNP detection, targeted sequencing, whole-genome amplification (WGA) and/or sequencing for aneuploidy analysis, insertion and deletion analysis of certain regions on the genes that have deleterious effects on the individual carrying such genetic defects, microarray based analysis for chromosomal abnormality (e.g., trisomy 18, 21 or 13).

The term “chromosomal abnormality” refers to a deviation between the structure of the subject chromosome and a normal homologous chromosome. The term “normal” refers to the predominate karyotype or banding pattern found in healthy individuals of a particular species. A chromosomal abnormality can be numerical or structural, and includes but is not limited to aneuploidy, polyploidy, inversion, a trisomy, a monosomy, duplication, deletion, deletion of a part of a chromosome, addition, addition of a part of chromosome, insertion, a fragment of a chromosome, a region of a chromosome, chromosomal rearrangement, and translocation. A chromosomal abnormality can be correlated with presence of a pathological condition or with a predisposition to develop a pathological condition. As defined herein, a single nucleotide polymorphism (“SNP”) is not a chromosomal abnormality.

Monosomy X (XO, absence of an entire X chromosome is the most common type of Turner syndrome, occurring in 1 in 2500 to 1 in 3000 live-born girls (Sybert and McCauley N Engl J Med (2004) 351:1227-1238). XXY syndrome is a condition in which human males have an extra X chromosome, existing in roughly 1 out of every 1000 males (Bock, Understanding Klinefelter Syndrome: A Guide for XXY Males and Their Families. NIH Pub. No. 93-3202 (1993)). XYY syndrome is an aneuploidy of the sex chromosomes in which a human male receives an extra Y chromosome, giving a total of 47 chromosomes instead of the more usual 46, affecting 1 in 1000 male births while potentially leading to male infertility (Aksglaede, et al., J Clin Endocrinol Metab (2008) 93:169-176).

Turner syndrome encompasses several conditions, of which monosomy X (XO, absence of an entire sex chromosome, the Barr body) is most common. Typical females have two X chromosomes, but in Turner syndrome, one of those sex chromosomes is missing. Occurring in 1 in 2000 to 1 in 5000 phenotypic females, the syndrome manifests itself in a number of ways. Klinefelter syndrome is a condition in which human males have an extra X chromosome. In humans, Klinefelter syndrome is the most common sex chromosome disorder and the second most common condition caused by the presence of extra chromosomes. The condition exists in roughly 1 out of every 1,000 males. XYY syndrome is an aneuploidy of the sex chromosomes in which a human male receives an extra Y chromosome, giving a total of 47 chromosomes instead of the more usual 46. This produces a 47, XYY karyotype. This condition is usually asymptomatic and affects 1 in 1000 male births while potentially leading to male infertility.

Trisomy 13 (Patau syndrome), trisomy 18 (Edward syndrome) and trisomy 21 (Down syndrome) are the most clinically important autosomal trisomies and how to detect them has always been the hot topic. Detection of above fetal chromosomal aberration has great significance in prenatal diagnosis (Ostler, Diseases of the eye and skin: a color atlas. Lippincott Williams & Wilkins. pp. 72. ISBN 9780781749992 (2004); Driscoll and Gross, N Engl Med (2009) 360: 2556-2562; Kagan, et al., Human Reproduction (2008) 23:1968-1975).

In some embodiments, analyzing a nucleotide sequence of a nucleic acid molecule comprises hybridizing a detectable probe to the genomic DNA of one or more isolated fetal cells. The approach may be that of FISH (fluorescence in situ hybridization). In sonic embodiments, analyzing a nucleotide sequence of a nucleic acid molecule comprises sequencing genomic DNA of one or more isolated fetal cells. In some embodiments, sequencing genomic DNA comprises sequencing the DNA of a single or few cells, and wherein sequencing the DNA of a single cell is performed for one or more isolated fetal cells.

It will he apparent to those skilled in the art that a number of different sequencing methods and variations can be used. In one embodiment, the sequencing is done using massively parallel sequencing. “Massively parallel sequencing” means techniques for sequencing millions of fragments of nucleic acids, e.g., using attachment of randomly fragmented genomic DNA to a planar, optically transparent surface and solid phase amplification to create a high density sequencing flow cell with millions of clusters, each containing ˜1,000 copies of template per sq. cm. These templates are sequenced using four-color DNA sequencing-by-synthesis technology. Massively parallel sequencing, such as that achievable on the 454 platform (Roche) (Margulies, et al., Nature (2005) 437:376-380), Illumina Genome Analyzer (or Solexa™ platform) or SOLiD System (Applied Biosystems) or the Helicos True Single Molecule DNA sequencing technology (Harris, et al., Science (2008) 320:106-109), the single molecule, real-time (SMRT™) technology of Pacific Biosciences, and nanopore sequencing (Soni and Meller, Clin Chem (2007) 53:1996-2001), allow the sequencing of many nucleic acid molecules isolated from a specimen at high orders of multiplexing in a parallel fashion (Dear, Brief Funct Genomic Proteomic (2003) 1:397-416). Each of these platforms sequences clonally expanded or even non-amplified single molecules of nucleic acid fragments. Commercially available sequencing equipment may be used in obtaining the sequence information of the polynucleotide fragments. The presently used sequencing is preferably carried out without a preamplification or cloning step, but may be combined with amplification-based methods in a microfluidic chip having reaction chambers for both PCR and microscopic template-based sequencing. Only about 30 bp of random sequence information are needed to identify a sequence as belonging to a specific human chromosome. Longer sequences can uniquely identify more particular targets. In the present case, a large number of 35 bp reads were obtained. Further description of a massively parallel sequencing method is found in Rogers and Ventner, Nature (2005) 437:326-327.

### Integrated Microfluidic Device

Embodiments disclosed herein provides integrated microfluidic devices for non-invasive isolation of fetal cells, comprising: a filter; a binding moiety or affinity molecule that specifically binds to a fetal cell-specific antigen or a non-fetal cell-specific antigen for positive selection of fetal cells or negative selection of unwanted cells; and a microscope based-visualizable chamber.

In some embodiments, the integrated microfluidic devices may be configured to be visualized on a microscope platform. For example, in some embodiments, the integrated microfluidic devices may comprise a filter that is transparent and visualizable under a microscope. In some embodiments, the integrated microfluidic devices may comprise a micro-channel and/or chamber that is transparent and visualizable under a microscope.

In some embodiments, the integrated microfluidic devices may comprise of multiple layers made from similar or dissimilar materials and assembled via various available bonding techniques.

In some embodiments, the integrated microfluidic devices may comprise a pump that is configured to drive fluid flow from a blood container to a micro-channel or chamber on the integrated microfluidic device. In some embodiments, the integrated microfluidic devices may comprise a pump that is configured to regulate fluid flow in the micro-channels and/or chambers on the microfluidic device using a combination of flexible membrane to act as a valve to regulate liquid flow to different areas of the chip.

In some embodiments, the integrated microfluidic devices may include a microvalve at an intersection between the micro-channels and/or chambers to control fluid flow. In some embodiments, more than one microvalve may be formed between the micro-channels and/or chambers at multiple intersections. In some embodiments, the microvalve may be controlled by a control channel. To activate the microvalves, the open end of the control channel may be connected to a pressure source, such as a pump, a syringe, a high-pressure cylinder. In certain embodiments, the liquid flow may be guided by vacuum.

In some embodiments, more than one control channel may be included in the microfluidic device. In embodiments wherein more than one control channel are included in the microfluidic device, each of the control channels may be operated together or separately. For example, some of the control channels may be pressurized while others are released of the pressure. In certain embodiments, operating the control channels separately may permit a sample and/or reagent to be added to some reaction chambers but not others.

Any suitable material may be used for the flexible membrane. For example, the materials of the elastic membrane can be PDMS, silicon rubber, memory alloy, or PTFE (polytetrafluoroethylene), etc., or a combination thereof.

Exemplary microfluidic devices may comprise a central body structure in which various microfluidic elements are disposed. The body structure includes an exterior portion or surface, as well as an interior portion which defines the various microscale channels and/or chambers of the overall microfluidic device. For example, the body structure of an exemplary microfluidic devices typically employs a solid or semi-solid substrate that may be planar in structure, i.e., substantially flat or having at least one flat surface. Suitable substrates may be fabricated from any one of a variety of materials, or combinations of materials. Often, the planar substrates are manufactured using solid substrates common in the fields of microfabrication, e.g., silica-based substrates, such as glass, quartz, silicon or polysilicon, as well as other known substrates, i.e., gallium arsenide. In the case of these substrates, common microfabrication techniques, such as photolithographic techniques, wet chemical etching, micromachining, i.e., drilling, milling and the like, may be readily applied in the fabrication of microfluidic devices and substrates. Alternatively, polymeric substrate materials may be used to fabricate the devices of the present invention, including, e.g., polydimethylsiloxanes (PDMS), polymethylmethacrylate (PMMA), polyurethane, polyvinylchloride (PVC), polystyrene, polysulfone, polycarbonate and the like. In the case of such polymeric materials, injection molding or embossing methods may be used to form the substrates having the channel and reservoir geometries as described herein. In such cases, original molds may be fabricated using any of the above described materials and methods. The assembled chips may be treated with plasma to alter surface wet-ability where desired post assembly or preferably treated first and then assembled.

### Kits

Embodiments disclosed herein further provide kits comprising: an integrated microfluidic device for non-invasive isolation of fetal cells, comprising: a filter; a binding moiety or affinity molecule that specifically binds to a fetal cell-specific antigen or a non-fetal cell-specific antigen; and optically clear chamber allowing for microscope visualization for confirmation, and a reagent that is configured to detect one or more nucleotide and/or polypeptide sequences of the isolated fetal cells.

Any suitable reagents can be used, for example, gel electrophoresis reagents, chromatography reagents, spectrophotometry reagents, etc., for detecting one or more nucleotide and/or polypeptide sequences of the isolated fetal cells. In some embodiments, the reagents for detecting one or more nucleotide and/or polypeptide sequences of the isolated fetal cells may be a sequencing device.

In some embodiments, the kits may comprise primers and primer pairs, which allow the specific amplification of the polynucleotides, and probes that selectively or specifically hybridize to nucleic acid molecules of the isolated fetal cells. Probes may be labeled with a detectable marker, such as, for example, fluorescent compound, bioluminescent compound, a chemiluminescent compound, metal chelator or enzyme. Such probes and primers can be used to detect the presence of polynucleotides in the isolated fetal cells.

In some embodiments, the kits may comprise reagents for detecting presence of polypeptides. Such reagents may be antibodies or other binding molecules that specifically bind to a polypeptide. In some embodiments, such antibodies or binding molecules may be capable of distinguishing a structural variation to the polypeptide as a result of polymorphism, and thus may be used for genotyping. The antibodies or binding molecules may be labeled with a detectable marker, such as, for example, a radioisotope, a fluorescent compound, a bioluminescent compound, a chemiluminescent compound, a metal chelator, an enzyme, or a particle. Other reagents for performing binding assays, such as ELISA, may be included in the kits.

In some embodiments, the kits comprise reagents for genotyping at least two, at least three, at least five, at least ten, or fifteen biomarkers. In some embodiments, the kits may further comprise a surface or substrate (such as a microarray) for capture probes for detecting of amplified nucleic acids.

The kits may further comprise a carrier means being compartmentalized to receive in close confinement one or more container means such as vials, tubes, and the like, each of the container means comprising one of the separate elements to he used in the method. For example, one of the container means may comprise a probe that is or can be detectably labeled. Such probe may be a polynucleotide specific for a biomarker. Where the kit utilizes nucleic acid hybridization to detect the target nucleic acid, the kit may also have containers containing nucleotide(s) for amplification of the target nucleic acid sequence and/or a container comprising a reporter-means, such as a biotin-binding protein, such as avidin or streptavidin, bound to a reporter molecule, such as an enzymatic, florescent, or radioisotope label.

The kits will typically comprise the container described above and one or more other containers comprising materials desirable from a commercial and user standpoint, including buffers, diluents, filters, needles, syringes, and package inserts with instructions for use. A label may be present on the container to indicate that the composition is used for a specific therapy or non-therapeutic application, and may also indicate directions for either in vivo or in vitro use, such as those described above.

The kits can further comprise a set of instructions and materials for preparing a tissue or cell sample and preparing nucleic acids (such as genomic DNA) from the sample.

The embodiments disclosed herein provide a variety of compositions suitable for use in performing methods of the invention, which may be used in kits. For example, the embodiments disclosed herein provide surfaces, such as arrays that can be used in such methods. In some embodiments, an array of the invention comprises individual or collections of nucleic acid molecules useful for detecting biomarkers of the invention. For instance, an array of the invention may comprises a series of discretely placed individual nucleic acid oligonucleotides or sets of nucleic acid oligonucleotide combinations that are hybridizable to a sample comprising target nucleic acids, whereby such hybridization is indicative of genotypes of the biomarkers of the invention.

Several techniques are well-known in the art for attaching nucleic acids to a solid substrate such as a glass slide. One method is to incorporate modified bases or analogs that contain a moiety that is capable of attachment to a solid substrate, such as an amine group, a derivative of an amine group or another group with a positive charge, into nucleic acid molecules that are synthesized. The synthesized product is then contacted with a solid substrate, such as a glass slide, which is coated with an aldehyde or another reactive group which will form a covalent link with the reactive group that is on the amplified product and become covalently attached to the glass slide.

### EXAMPLE

Maternal blood sample is collected from a pregnant women in 1^{st} or early second trimester of pregnancy. The blood sample is processed within 48 hours after collection. The blood sample is diluted 1:2-1:20 with phosphate-buffered saline (PBS) to appropriate volume. The diluted blood sample is applied to a microfluidic chip with an integrated filter through syringe or automated pipet like system. The filter has a smallest effective opening of 5 μm and various other embodiments in terms of opening patterns and structure. After the blood sample passes through the filter, typically 2 mL of washing buffer is applied to the microfluidic chip at room temperature for 3 times. The enriched nucleated blood cells are labeled with DAPI by applying a staining solution through a micro-channel connected to a solution reservoir by a conduit. Fetal cells identified via various fluorescent labeling strategies which include labeling with Fitc or PE conjugated anti-CD71 antibody and or anti-GPA antibody. A significant portion of maternal nucleated blood cells are removed or rejected via staining with fluorescent tagged CD45 antibody. The labeled fetal cells are visualized by placing the chip on a microscopic platform. A video camera is used to capture fluorescent images of the labeled fetal cells either via direct illumination from top or via epi-illumination from the bottom (the exact nature of this configuration is still being decided upon).

10 mg of protein A/G magnetic beads mixture (Life Technologies Corporation) are suspended and washed twice with PBS plus Tween-20. 50 μg of CD45 antibodies are added to 400 μl beads mixture diluted in PBS plus Tween-20, incubated and rotated at room temperature for 30 min. Beads coupled with CD45 antibody are separated with magnetic stand and washed for 3 times in PBS plus Tween-20. CD45 targets leukocytes and removes a significant portion of the non-fetal population of the collected blood sample reducing cell load for detection and isolation and increasing throughput. Antibody-coated beads are added to the enriched nucleated blood cells and incubated for 30 min. The non-fetal cells attached to the beads are removed from the fetal cells by applying a magnetic force to the chip to move the beads to a collection chamber on the chip. This step can also be carried out as an earlier step off-chip for negative depletion prior to loading the partially enriched blood sample on chip.

The top layer of the chip is separated from the bottom layer, exposing the chamber containing the labeled fetal cells. An automated robotic pipette is used to isolate the fetal cells into a micro titer plate controlled by a computer using fluorescent images obtained by a video camera. Genomic DNA is purified from the collected fetal cells and analyzed for trisomy 21, 18 or 13 or other genetic anomalies by Next Generation Sequencing (NGS) methods, microarray analysis, FISH or SNP based genomic analysis.

1. An method for isolation of fetal cells for non-invasive prenatal diagnosis, comprising:
providing a maternal blood sample;
applying the maternal blood sample to a filter integrated on a microfluidic device to thereby enrich the nucleated blood cells from the maternal blood sample;
labeling the enriched nucleated blood cells, within the microfluidic device, with a fluorescent binding moiety or affinity molecule that specifically binds to a fetal cell-specific antigen or a non-fetal cell-specific antigen; and
isolating the fetal cells.

2. The method of claim 1, wherein the filter is transparent.

3. The method of claim 1, wherein the nucleated blood cells are enriched via morphology and/or other physical characteristics of the cells.

4. The method of claim 1, further comprising visualizing the labeled nucleated blood cells in a microscopy-visualizable chamber within the microfluidic device.

5. The method of claim 4, further comprising selectively immobilizing labeled nucleated blood cells within the filter fitted microfluidic for visualization and/or microscopic analysis, and wherein the visualization and/or microscopic analysis is manual or automated via machine vision.

6. (canceled)

7. (canceled)

8. The method of claim 1, wherein the fetal cells are nucleated red blood cells (nRBCs), and wherein the fetal cell-specific antigen is selected from the group consisting of CD45, transferrin receptor (CD71), glycophorin A (GPA), HLA-G, EGFR, thrombospondin receptor (CD36), CD34, HbF, HAE 9, FB3-2, H3-3, erythropoietin receptor, HBE, AFP, APOC3, SERPINC1, AMBP, CPB2, ITIH1, APOH, HPX, beta-hCG, AHSG, APOB, J42-4-d, 2,3-biophosphoglycerate (BPG), Carbonic anhydrase (CA), Thymidine kinase (TK), MMP14 (matrix metalloproteinase 14), and fetal hemoglobin.

9. (canceled)

10. The method of claim 1, wherein the filter is configured to enrich nucleated blood cells and-'or remove mature red blood cells (RBCs).

11. The method of claim 1, further comprising removing non-fetal cells.

12. The method of claim 11, wherein the removing non-fetal cells comprises immobilizing the non-fetal cells.

13. The method of claim 1, comprising immobilizing the fetal cells.

14. The method of claim 13, wherein immobilizing the fetal cells comprises contacting the fetal cells with a binding moiety or affinity molecule that specifically binds to a fetal cell-specific antigen, and wherein the fetal cell-specific antigen is selected form the group consisting of CD45, transferrin receptor (CD71), glycophorin A (GPA), HLA-G, EGFR, thrombospondin receptor (CD36), CD34, HbF, HAE 9, FB3-2, H3-3, erythropoietin receptor, HBE, AFP, APOC3, SERPINC1, AMBP, CPB2, ITIH1, APOH, HPX, beta-hCG, AHSG, APOB, J42-4-d, 2,3-biophosphoglycerate (BPG), Carbonic anhydrase (CA), Thymidine kinase (TK MMP14 (matrix metalloproteinase 14), and fetal hemoglobin.

15. (canceled)

16. The method of claim 1, further comprising analyzing the isolated fetal cell for nucleic acid sequence for a genetic defect via sequencing or detecting genetic abnormalities in the isolated fetal cell using FISH or DNA microarray.

17. The method of claim 16, wherein the analyzing a nucleotide sequence of a nucleic acid molecule comprises at least one of:
(a) hybridizing a detectable probe to the genomic DNA of one or more isolated fetal cells, and
(b) sequencing genomic DNA of one or more isolated fetal cells.

18. (canceled)

19. The method of claim 17, wherein the sequencing genomic DNA comprises sequencing the DNA of a single cell, and wherein sequencing the DNA of a single cell is performed for one or more isolated fetal cells.

20. The method of claim 16, wherein the analyzing expression of a gene comprises hybridizing a detectable antibody to the surface of one or more isolated fetal cells.

21. (canceled)

22. An integrated microfluidic device for non-invasive isolation of fetal cells, comprising:
a filter;
a binding moiety or affinity molecule that specifically binds to a fetal cell-specific antigen or a non-fetal cell-specific antigen; and
a microscopy-visualizable chamber.

23. The integrated microfluidic device of claim 22, comprising a reagent that is configured to detect one or more nucleotide sequences of the isolated fetal cells.

24. A kit comprising:
an integrated microfluidic device for non-invasive isolation of fetal cells, comprising:
a filter;
a binding moiety or affinity molecule that specifically binds to a fetal cell-specific antigen or a non-fetal cell-specific antigen; and
a microscopy-visualizable chamber, and
a reagent that is configured to detect one or more nucleotide sequences of the isolated fetal cells.

25. The integrated microfluidic device of claim 22, further comprising a pump that is configured to drive fluid flow from a blood container to a micro-channel or chamber on the integrated microfluidic device.

26. The integrated microfluidic device of claim 25, further comprising a microvalve at an intersection between the micro-channels and/or chambers to control fluid flow.
